(19) United States
(12) Patent Application Publication
(10) Pub. No.: US 2010/0322961 A1
(43) Pub. Date: Dec. 23, 2010

(54) Intranasal Drug Delivery System

(76) Inventor: Wayne Jeffrey Perry, Albany, NY (US)

Correspondence Address: SUZANNAH K. SUNDBY;SMITH, GAMBRELL & RUSSEL, LLP, 1130 Connecticut Avenue, NW, Suite 1130, WASHINGTON, DC, 20036, US

(21) Appl. No.: 12/849,088

(22) Filed: Aug. 3, 2010

Related U.S. Application Data
(63) Continuation of application No. 11/731,657, filed on Mar. 30, 2007, now abandoned.

(51) Int. Cl. A61K 36/06; A61K 31/16; A61K 36/534; A61K 36/752; A61K 36/82; A61K 36/258

(52) U.S. Cl. 424/195.15; 514/625; 514/627; 424/747; 424/736; 424/729; 424/728

(57) ABSTRACT

The present invention provides an intranasal drug delivery system containing capsaicin, dihydrocapsaicin, nordihydrocapsaicin, homodihydrocapsaicin, and homocapsaicin in the form of oleoresin capsicum used as a carrier to quickly and effectively deliver a drug.

### FIELD OF THE INVENTION

The present invention relates to an intranasal drug delivery system containing capsaicinoids used as a carrier to quickly and effectively deliver a drug. The present invention is related to a copending application entitled SINUS RELIEF COMPOSITION AND METHOD OF PRODUCING THE SAME filed by the inventor with the United States Patent Office and which disclosure is incorporated herein.

### BACKGROUND OF THE INVENTION

Capsaicinoids are the active components in chili peppers and plants belonging to the Capsicum family. The pungency level of a plant belonging to the Capsicum family is measured on the Scoville scale. The greater the number of Scoville heat units indicates the “hotter” the plant. Capsaicin is a capsaicinoid and the most prevalent capsaicinoid in chili peppers, followed by dihydrocapsaicin, nordihydrocapsaicin, homodihydrocapsaicin, and homocapsaicin. Pure capsaicin ranges between 15,000,000-16,000,000 Scoville units. Dihydrocapsaicin is an irritant and has a similar pungency to capsaicin. Nordihydrocapsaicin, homodihydrocapsaicin, and homocapsaicin are also irritants and have a pungency of about 8,600,000-9,100,000 Scoville units.

Each capsaicinoid and its corresponding chemical structure is shown below.

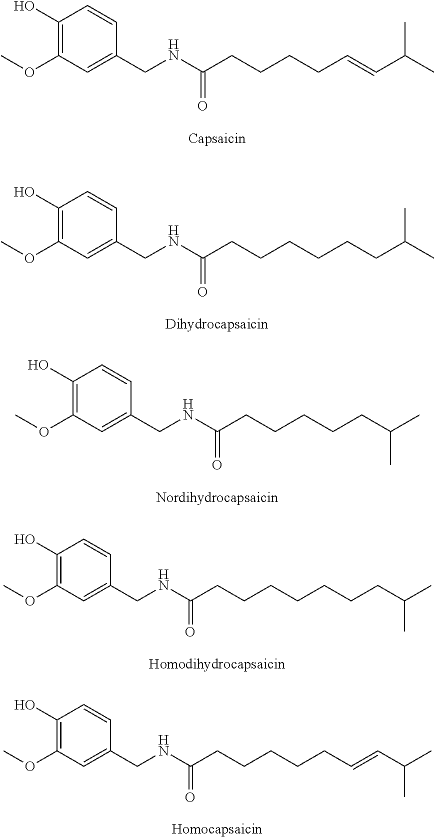

Capsaicinoids are irritants and produce a sensation of burning or hotness when they come in contact with human tissue. Capsaicinoids chemically interact with sensory neurons by binding to thermoreceptor nerve endings inside the body, or to receptors in the skin. These receptors may be stimulated with heat or physical abrasion causing chemical signals to pass through the cell membrane and into the cell which causes the neuron to generate its own signal to the brain. The capsaicinoid molecules induce the same effect of producing a burning sensation, but not a chemical burn, that heat or physical abrasion does by binding to the thermoreceptor nerve endings.

Capsaicinoids have many uses such as a heat additive for foods, topical ointments, and non-lethal weapons. Capsaicinoids are used in foods to add spice or “heat”, such as in hot sauce or salsa. Capsaicinoids are also used as a topical ointment to relieve minor aches and pains in muscles and joints, such as symptoms associated with arthritis. Capsaicinoids are also used as an ingredient in the non-lethal weapon commonly known as “pepper spray”, which when sprayed into the eyes or onto the skin is painful to the recipient.

At present, drug delivery systems, including intranasal systems, present delivery problems such as low absorption and low effectiveness and efficiency in delivering drugs past the blood-brain barrier to the brain and throughout the central nervous system. The permeability of drugs past the barrier to the brain and throughout the central nervous system is increased by receptor mediated peptides that respond to very few chemicals. Intranasal drug delivery systems are generally utilized only for treatment of local ailments, such as cold, cough, and allergies and not utilized for systemic drug delivery. Systemic intranasal drug delivery is limited by molecular weight, size, shape, formulation pH, delivery volume, and the inability to maintain the effectiveness of the drug once it is delivered. Intranasal drug delivery, however, is advantageous because it avoids pain during administration, such as with needle drug delivery systems.

### SUMMARY OF THE INVENTION

Thus, one object of the present invention is a natural intranasal drug delivery system.

Another object of the present invention is an intranasal drug delivery system which increases absorption, while efficiently, quickly and effectively delivering a therapeutic or diagnostic drug.

A further object of the present invention is an intranasal drug delivery system which increases the permeability of a drug beyond the barriers to the brain and central nervous systems.

Still another object of the present invention is an intranasal drug delivery system which will not degrade the drug and will not contradict with the drug actives.

In accordance with the present invention, a natural drug delivery system containing capsaicin, dihydrocapsaicin, nordihydrocapsaicin, homodihydrocapsaicin, and homocapsaicin is utilized to increase the permeability and the absorption of a drug, while efficiently, quickly and effectively delivering the drug to the patient. In one form, the drug delivery system has been developed for intranasal administration of a liquid drug.

The intranasal drug delivery system in accordance with the present invention, preferably, includes oleoresin capsicum including capsaicin, dihydrocapsaicin, nordihydrocapsaicin, homodihydrocapsaicin, and homocapsaicin that has been developed specifically for the purpose of increasing permeability and absorption of a drug.

In one embodiment of the present invention, the intranasal drug delivery system includes between 0.000001% to 0.0071% by weight of the total water or suspension material weight of oleoresin capsicum including capsaicin, dihydrocapsaicin, nordihydrocapsaicin, homodihydrocapsaicin, and homocapsaicin, as a carrier for a drug.

In another embodiment of the present invention, the intranasal drug delivery system includes between 1 ppb to 50,000 ppb oleoresin capsicum including capsaicin, dihydrocapsaicin, nordihydrocapsaicin, homodihydrocapsaicin, and homocapsaicin, as a carrier for a drug.

In another embodiment of the present invention, the intranasal drug delivery system includes oleoresin capsicum, including capsaicin, dihydrocapsaicin, nordihydrocapsaicin, homodihydrocapsaicin, and homocapsaicin, as a carrier for a drug and having a heat range of 100,000 to 500,000 Scoville units.

In yet another embodiment of the present invention, the sinus relief composition includes oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin.

In a further embodiment of the present invention, the oleoresin capsicum may be water soluble.

Additionally, in the method of delivering a drug using the intranasal drug delivery system of the present invention, the oleoresin capsicum, including capsaicin, dihydrocapsaicin, nordihydrocapsaicin, homodihydrocapsaicin, and homocapsaicin, is a carrier for the drug. These and other embodiments of the present invention are more fully described in connection with the detailed description.

### DETAILED DESCRIPTION OF THE INVENTION

The present invention relates to an intranasal drug delivery system containing capsaicin, dihydrocapsaicin, nordihydrocapsaicin, homodihydrocapsaicin, and homocapsaicin used as a carrier to quickly and effectively deliver a drug. The present invention further relates to an intranasal drug delivery system that has been developed to increase absorption and permeability of a drug. The present drug delivery system is designed for the intranasal administration of a liquid drug.

Oleoresin capsicum is a natural resinous extract derived from several Capsicum pepper varieties. The Capsicum variety utilized in the present invention is derived from cayenne pepper plants, which includes Capsicum baccatum and Capsicum frutescens. The oleoresin capsicum includes capsaicin, dihydrocapsaicin, nordihydrocapsaicin, homodihydrocapsaicin, and homocapsaicin as active ingredients. Preferably, the oleoresin capsicum utilized in the present invention is an all natural, water soluble or soluble liquid suspension material. The use of natural oleoresin capsicum including capsaicin, dihydrocapsaicin, nordihydrocapsaicin, homodihydrocapsaicin, and homocapsaicin as opposed to synthetic capsaicin allows for repeated use and effective delivery without too much heat. However, it is within the scope of the present invention that a combination of synthetic capsaicinoids including about 67-71% capsaicin, about 20-24% dihydrocapsaicin, about 5-9% nordihydrocapsaicin, about 0.25-2% homodihydrocapsaicin, and about 0.25-2% homocapsaicin could also be used.

Due to the combination of the capsaicinoids, the oleoresin capsicum increases the absorption and permeability of a drug. Through intranasal administration, the oleoresin capsicum facilitates the absorption of a drug because the capsaicinoids allow the drug to bind to the nasal membranes and nerves for longer periods of time than when capsaicinoids are not present, and therefore, increases absorption of the drug. A recipient's body also contains certain peptides called receptor mediated permeabilizers (RMPs) which increase the permeability of drugs past the blood-brain barrier. The oleoresin capsicum increases the permeability of a drug beyond the blood-brain barrier because the capsaicinoids release a variety of peptides upon contact with the nerve fibers and other membranes, specifically throughout the Trigeminal nerve network. The oleoresin capsicum also increases delivery and efficiency of a drug throughout the central nervous system and the bloodstream.

In one embodiment of the present invention, the intranasal drug delivery system may include between 0.000001% to 0.0071% by weight of the total water or suspension material weight of oleoresin capsicum including capsaicin, dihydrocapsaicin, nordihydrocapsaicin, homodihydrocapsaicin, and homocapsaicin as a carrier for a drug.

In another embodiment of the present invention, the intranasal drug delivery system includes between 1 ppb to 50,000 ppb oleoresin capsicum including capsaicin, dihydrocapsaicin, nordihydrocapsaicin, homodihydrocapsaicin, and homocapsaicin, as a carrier for a drug.

In another embodiment of the present invention, the intranasal drug delivery system may include oleoresin capsicum including capsaicin, dihydrocapsaicin, nordihydrocapsaicin, homodihydrocapsaicin, and homocapsaicin as a carrier for a drug, and having a heat range of between 100,000 to 500,000 Scoville units. The oleoresin capsicum including capsaicin, dihydrocapsaicin, nordihydrocapsaicin, homodihydrocapsaicin, and homocapsaicin is present in a non-caustic and safe amount.

In yet another embodiment of the present invention, the sinus relief composition includes oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin.

In still another embodiment of the present invention, the oleoresin capsicum may be water soluble.

Additionally, the drug can be a therapeutic agent or a diagnostic agent. In a further embodiment of the present invention, less amounts of the drug are required as compared to a drug delivered without capsaicinoids due to the increased permeability and absorption from the capsaicinoids as the carrier of the drug.

Also, in one embodiment of the present invention, the drug can be natural or synthesized, or any combination thereof and in another embodiment, the oleoresin capsicum including capsaicin, dihydrocapsaicin, nordihydrocapsaicin, homodihydrocapsaicin, and homocapsaicin can be an active ingredient and a carrier of a drug.

In still another embodiment of the present invention, the intranasal drug delivery system may be an intranasal spray. In a further embodiment of the present invention, the intranasal drug delivery system may be an intranasal spray wherein a drug is released in a metered dose. In yet another embodiment of the present invention, the intranasal drug delivery system may be an intranasal spray wherein a drug is released in a time released dose. This administration route facilitates the drug to more quickly enter the bloodstream, to permeate past the blood-brain barrier, and enter the central nervous system and bloodstream simultaneously.

The intranasal drug delivery system can be an intranasal spray for delivering a drug wherein the intranasal spray is inserted into a first nostril of a human. A second nostril may be held closed by the human. The intranasal drug delivery system is sprayed between one to three times into the first nostril. The intranasal drug delivery system may be sniffed into the nostril as far into the nostril as it can be sniffed. Then the process is repeated for the second nostril. The process may be repeated two to three times per day for both nostrils until the symptoms subside or relief is provided. If the symptoms are severe, then the drug can be administered through an intranasal spray as needed.

One embodiment of the invention also relates to a method of delivering a drug using the intranasal drug delivery system described above and wherein oleoresin capsicum including capsaicin, dihydrocapsaicin, nordihydrocapsaicin, homodihydrocapsaicin, and homocapsaicin is a carrier for the drug. The method includes the intranasal administration of a drug with oleoresin capsicum including capsaicin, dihydrocapsaicin, nordihydrocapsaicin, homodihydrocapsaicin, and homocapsaicin as a carrier for the drug. The method of delivering the drug increases absorption and permeability of a drug by using previously noted capsaicinoids as the carrier. In still another embodiment of the method of delivering a drug, the drug may permeate beyond the blood-brain barrier due to the previously noted capsaicinoids as the carrier for the drug. In another embodiment of the method of delivering a drug, the drug can be absorbed more efficiently throughout the central nervous system through the use of previously noted capsaicinoids as the carrier for the drug.

The following are examples of formulations including a drug and the intranasal drug delivery system described above wherein the capsaicinoids of the intranasal drug delivery system provide quick, effective, and increased absorption and permeability of the drug. It will be apparent to one skilled in the art that these are examples of formulations utilizing the intranasal drug delivery system and many more formulations are possible.

### EXAMPLE 1

One example of the present invention relates to a headache relief composition for use with the intranasal drug delivery system. The headache relief composition provides relief of symptoms such as migraines, general headaches, chronic and occasional headaches, and dizziness, and visual distortions associated with headaches. The headache relief composition includes oleoresin capsicum including capsaicin, dihydrocapsaicin, nordihydrocapsaicin, homodihydrocapsaicin, and homocapsaicin as the intranasal drug delivery system; feverfew extract, eucalyptol, and peppermint oil may comprise the drug; and grapefruit seed extract, rosemary extract, vegetable glycerin, ascorbic acid, citric acid, and sea salt as other components.

The feverfew extract relieves and prevents headache symptoms. The eucalyptol is a decongestant which clears nasal congestion and relieves sinus and allergy symptoms. The peppermint oil relieves and prevents headache symptoms. The grapefruit seed extract is an anti-bacterial agent and preservative. The rosemary extract is an anti-microbial agent and a natural preservative which protects and stabilizes the headache relief composition. The vegetable glycerin is a moisturizer, assists the capsaicinoids contained in the oleoresin capsicum to maintain its potency and effectiveness for longer periods of time, shortens the length of time of the burning sensation associated with the capsaicinoids in the oleoresin capsicum without reducing its effectiveness, and stabilizes the formula. The ascorbic acid adjusts the pH supports the immune system, and acts as a natural preservative. The citric acid further adjusts the pH level and stabilizes the formula. The sea salt acts as a nasal cavity cleanser which flushes out bacteria, and dried or clogged mucous which can affect the performance of nerve receptors in the trigeminal region.

In another embodiment of the present invention, the headache relief composition may be homeopathic wherein the eucalyptol is for congestion and dryness in the throat, and the feverfew extract is for headache relief. Furthermore, the headache relief composition may include both eucalyptol and feverfew extract as a tincture.

In still another embodiment of the present invention, the headache relief composition may include between about 0.0044% to 0.0047% by weight of the total water weight of oleoresin capsicum including capsaicin, dihydrocapsaicin, nordihydrocapsaicin, homodihydrocapsaicin, and homocapsaicin for use with the intranasal drug delivery system.

One example of the headache relief composition in accordance with the present invention for use with the intranasal drug delivery system in 5 gallons of purified water includes:

0.0044% to 0.0047% by weight of the total water weight of oleoresin capsicum;

0.10% by weight of the total water weight of feverfew extract;

0.0027% by weight of the total water weight of peppermint oil;

0.13% by weight of the total water weight of eucalyptol;

0.08% by weight of the total water weight of rosemary extract;

0.05% by weight of the total water weight of grapefruit seed extract;

3.65% by weight of the total water weight of vegetable glycerin;

0.53% by weight of the total water weight of sea salt;

0.83% by weight of the total water weight of ascorbic acid; and

0.26% by weight of the total water weight of citric acid.

In another embodiment, the headache relief composition may be administered as a preventative and symptomatic tool.

### EXAMPLE 2

Another example of the present invention relates to an allergy relief composition having the intranasal drug delivery system. The allergy relief composition provides relief of symptoms caused by allergies such as nasal congestion, sinus pressure, and headaches. The allergy relief composition includes oleoresin capsicum including capsaicin, dihydrocapsaicin, nordihydrocapsaicin, homodihydrocapsaicin, and homocapsaicin as the intranasal drug delivery system; nettle extract, and eucalyptol may comprise the drug; and grapefruit seed extract, rosemary extract, vegetable glycerin, ascorbic acid, citric acid and sea salt as other components.

The nettle extract relieves and desensitizes allergy symptoms and related allergy triggers. The eucalyptol is a decongestant which clears nasal congestion and relieves sinus and allergy symptoms. The grapefruit seed extract is an anti-bacterial agent and preservative. The rosemary extract is an anti-microbial agent and a natural preservative which protects and stabilizes the allergy relief composition. The vegetable glycerin is a moisturizer, assists the capsaicinoids contained in the oleoresin capsicum to maintain their potency and effectiveness for longer periods of time, shortens the length of time of the burning sensation associated with the capsaicinoids in the oleoresin capsicum without reducing its effectiveness, and stabilizes the formula. The ascorbic acid adjusts the pH level, supports the immune system, and acts as a natural preservative. The citric acid adjusts the pH level and stabilizes the formula. The sea salt acts as a nasal cavity cleanser which flushes out bacteria, and dried or clogged mucous therefrom.

In another embodiment of the present invention, the allergy relief composition may be homeopathic wherein the eucalyptol is for congestion, and dryness in the throat, and the nettle extract is for allergy relief. Furthermore, the homeopathic allergy relief composition may include eucalyptol as a tincture and nettle extract as a tincture.

In still another embodiment of the present invention, the allergy relief composition may include between about 0.0029% to 0.0032% by weight of the total water weight of oleoresin capsicum including capsaicin, dihydrocapsaicin, nordihydrocapsaicin, homodihydrocapsaicin, and homocapsaicin for use with the intranasal drug delivery system.

Another embodiment of the allergy relief composition having the intranasal drug delivery system in 5 gallons of purified water includes:

0.0029% to 0.0032% by weight of the total water weight of oleoresin capsicum;

0.10% by weight of the total water weight of nettle extract;

0.13% by weight of the total water weight of eucalyptol;

0.08% by weight of the total water weight of rosemary extract;

0.05% by weight of the total water weight of grapefruit seed extract;

3.65% by weight of the total water weight of vegetable glycerin;

0.53% by weight of the total water weight of sea salt;

0.83% by weight of the total water weight of ascorbic acid; and

0.26% by weight of the total water weight of citric acid.

In another embodiment, the allergy relief composition may be administered as a preventative and symptomatic tool.

### EXAMPLE 3

A further example of the present invention relates to a weight control composition for use with the intranasal drug delivery system. The weight control composition provides relief of symptoms caused by excessive hunger, slow metabolism, and inconsistent blood sugar levels. The weight control composition includes oleoresin capsicum including capsaicin, dihydrocapsaicin, nordihydrocapsaicin, homodihydrocapsaicin, and homocapsaicin as the intranasal drug delivery system; licorice root extract, green tea extract and Chinese ginseng extract may comprise the drug; and grapefruit seed extract, spearmint oil, vegetable glycerin, ascorbic acid, and citric acid as other components.

The licorice root extract strengthens adrenal glands and regulates blood sugar levels. The Chinese ginseng extract provides an increase in energy levels. The green tea extract provides an increase in metabolism and is an anti-oxidant. The grapefruit seed extract is an anti-bacterial agent and preservative. The spearmint oil provides a sweet taste and is a decongestant. The vegetable glycerin is a moisturizer, assists the capsaicinoids contained in the oleoresin capsicum to maintain their potency and effectiveness for longer periods of time, shortens the length of time of the burning sensation associated with the capsaicinoids in the oleoresin capsicum without reducing its effectiveness, and stabilizes the formula. The ascorbic acid adjusts the pH level, supports the immune system, and acts as a natural preservative. The citric acid adjusts the pH level and stabilizes the formula.

In another embodiment of the present invention, the weight control composition may be homeopathic wherein the licorice root extract strengthens the adrenal glands and regulates blood sugar levels. Furthermore, the homeopathic weight control composition may include licorice root extract as a tincture.

In still another embodiment of the present invention, the weight control composition may include between about 0.0018% to 0.0021% by weight of the total water weight of oleoresin capsicum including capsaicin, dihydrocapsaicin, nordihydrocapsaicin, homodihydrocapsaicin, and homocapsaicin for use with the intranasal drug delivery system.

Another embodiment of the weight control composition for use with the intranasal drug delivery system in 5 gallons of purified water includes:

0.0018% to 0.0021% by weight of the total water weight of oleoresin capsicum;

0.20% by weight of the total water weight of licorice root extract;

0.15% by weight of the total water weight of Chinese ginseng extract;

0.20% by weight of the total water weight of green tea extract;

0.025% by weight of the total water weight of spearmint oil;

0.05% by weight of the total water weight of grapefruit seed extract;

3.65% by weight of the total water weight of vegetable glycerin;

0.83% by weight of the total water weight of ascorbic acid; and

0.26% by weight of the total water weight of citric acid.

In another embodiment, the weight control composition may be administered before & during meals, before & during workouts, and whenever the user is hungry.

### EXAMPLE 4

Another example of the present invention relates to a cold relief composition for use with the intranasal drug delivery system. The cold relief composition prevents and provides relief of symptoms caused by colds, flu, and poor immune system performance. The cold relief composition includes oleoresin capsicum including capsaicin, dihydrocapsaicin, nordihydrocapsaicin, homodihydrocapsaicin, and homocapsaicin as the intranasal drug delivery system; echinacea extract, eucalyptol, and golden seal extract may comprise the drug; and green tea extract, grapefruit seed extract, spearmint oil, maitake mushroom extract, cats claw extract, vegetable glycerin, ascorbic acid, citric acid and sea salt as other components.

The echinacea extract and the golden seal extract support the immune system. The eucalyptol is a decongestant which clears nasal congestion and relieves cold symptoms. The green tea extract is an anti-oxidant for the prevention of colds. The grapefruit seed extract is an anti-bacterial agent and preservative. The spearmint oil provides a sweet taste. The maitake mushroom extract and the cats claw extract support the immune system. The vegetable glycerin is a moisturizer, assists the capsaicinoids contained in the oleoresin capsicum to maintain its potency and effectiveness for longer periods of time, shortens the length of time of the burning sensation associated with the capsaicinoids in the oleoresin capsicum without reducing its effectiveness, and stabilizes the formula. The ascorbic acid adjusts the pH level, supports the immune system, and acts as a natural preservative. The citric acid adjusts the pH level and stabilizes the formula. The sea salt acts as a natural preservative.

In another embodiment of the present invention, the cold relief composition may be homeopathic wherein the eucalyptol is for congestion and dryness in the throat, and the echinacea extract and golden seal extract provide immune system support. Furthermore, the homeopathic cold relief composition may include eucalyptol as a tincture, echinacea extract as a tincture, and golden seal extract as a tincture.

In still another embodiment of the present invention, the cold relief composition may include between about 0.0024% to 0.0027% by weight of the total water weight of oleoresin capsicum including capsaicin, dihydrocapsaicin, nordihydrocapsaicin, homodihydrocapsaicin, and homocapsaicin for use with the intranasal drug delivery system.

One example of the cold relief composition in accordance with the present invention for use with the intranasal drug delivery system in 5 gallons of purified water includes:

0.0024% to 0.0027% by weight of the total water weight of oleoresin capsicum;

0.20% by weight of the total water weight of echinacea extract;

0.13% by weight of the total water weight of eucalyptol;

0.15% by weight of the total water weight of golden seal extract;

0.10% by weight of the total water weight of green tea extract;

0.10% by weight of the total water weight of cats claw extract;

0.013% by weight of the total water weight of spearmint oil;

0.05% by weight of the total water weight of grapefruit seed extract;

0.20% by weight of the total water weight of maitake mushroom extract;

0.53% by weight of the total water weight of sea salt;

3.65% by weight of the total water weight of vegetable glycerin;

0.83% by weight of the total water weight of ascorbic acid; and

0.26% by weight of the total water weight of citric acid.

In another embodiment, the cold relief composition may be administered before coming into contact with potential germs such as crowded environments, malls, schools, and airplanes.

### EXAMPLE 5

Another example of the present invention relates to an anti-smoking composition for use with the intranasal drug delivery system. The anti-smoking composition provides relief of symptoms caused by nicotine withdrawal such as headaches, anxiousness, decreased energy, excessive hunger, and mucous build-up. The anti-smoking composition includes oleoresin capsicum including capsaicin, dihydrocapsaicin, nordihydrocapsaicin, homodihydrocapsaicin, and homocapsaicin as the intranasal drug delivery system; marshmallow root extract and kava extract may comprise the drug; and guarana seed extract, grapefruit seed extract, spearmint oil, vegetable glycerin, ascorbic acid, and citric acid as other components.

The marshmallow root extract provides the taste of a cigarette to satisfy the taste buds. The kava extract calms the nerves and relieves anxiety caused by nicotine withdrawal. The guarana seed extract provides an increase in energy and concentration. The grapefruit seed extract is an anti-bacterial agent and preservative. The spearmint oil provides a sweet taste and is a decongestant. The vegetable glycerin is a moisturizer, assists the capsaicinoids contained in the oleoresin capsicum to maintain its potency and effectiveness for longer periods of time, shortens the length of time of the burning sensation associated with the capsaicinoids in the oleoresin capsicum without reducing its effectiveness, and stabilizes the formula. The ascorbic acid adjusts the pH level, supports the immune system, and acts as a natural preservative. The citric acid adjusts the pH level and stabilizes the formula.

In another embodiment of the present invention, the anti-smoking composition may be homeopathic wherein the marshmallow root extract provides the taste of a cigarette to satisfy the taste buds and the kava extract calms the nerves and relieves anxiety caused by nicotine withdrawal. Furthermore, the homeopathic anti-smoking composition may include marshmallow root extract as a tincture and kava extract as a tincture.

In still another embodiment of the present invention, the anti-smoking composition may include between about 0.0029% to 0.0032% by weight of the total water weight of oleoresin capsicum including previously noted capsaicinoids for use with the intranasal drug delivery system.

Another embodiment of the anti-smoking composition for use with the intranasal drug delivery system in 5 gallons of purified water includes:

0.0029% to 0.0032% by weight of the total water weight of oleoresin capsicum;

0.10% by weight of the total water weight of marshmallow root extract;

0.15% by weight of the total water weight of kava extract;

0.026% by weight of the total water weight of spearmint oil;

0.05% by weight of the total water weight of grapefruit seed extract;

3.65% by weight of the total water weight of vegetable glycerin;

0.83% by weight of the total water weight of ascorbic acid; and

0.26% by weight of the total water weight of citric acid.

In another embodiment of the present invention, the anti-smoking composition may be administered at least five times per day and whenever nicotine is craved, such as, after a meal, when drinking alcohol, and during “coffee breaks”.

### EXAMPLE 6

Another example of the present invention relates to a menstrual relief composition for use with the intranasal drug delivery system. The menstrual relief composition provides relief of symptoms caused by menstruation such as headaches, bloating, cramps, mood swings, and hot flashes. The menstrual relief composition includes oleoresin capsicum including capsaicin, dihydrocapsaicin, nordihydrocapsaicin, homodihydrocapsaicin, and homocapsaicin as the intranasal drug delivery system; chaste berry, wild yam root, fennel, and passion flower as the drug; and don quai extract, ascorbic acid, vegetable glycerin, citric acid, grapefruit seed extract, and spearmint oil as other components.

The chaste berry, wild yam root, fennel, and passion flower provide relief from symptoms related to menstruation. The don quai extract is an anti-oxidant. The grapefruit seed extract is an anti-bacterial agent and preservative. The spearmint oil provides a sweet taste. The vegetable glycerin is a moisturizer, assists the capsaicin, dihydrocapsaicin, nordihydrocapsaicin, homodihydrocapsaicin, and homocapsaicin contained in the oleoresin capsicum to maintain its potency and effectiveness for longer periods of time, shortens the length of time of the burning sensation associated with the capsaicinoids in the oleoresin capsicum without reducing its effectiveness, and stabilizes the formula. The ascorbic acid adjusts the pH level, supports the immune system, and acts as a natural preservative. The citric acid adjusts the pH level and stabilizes the formula.

In another embodiment of the present invention, the menstrual relief composition may be homeopathic wherein the chaste berry, wild yam root, fennel, and passion flower provide relief from symptoms related to menstruation. Furthermore, the homeopathic menstrual relief composition may include chaste berry, wild yam root, fennel and passion flower as tinctures.

In still another embodiment of the present invention, the menstrual relief composition may include between about 0.0010% to 0.0013% by weight of the total water weight of oleoresin capsicum including capsaicin, dihydrocapsaicin, nordihydrocapsaicin, homodihydrocapsaicin, and homocapsaicin for use with the intranasal drug delivery system.

One example of the menstrual relief composition for use with the intranasal drug delivery system in 5 gallons of purified water includes:

0.0010% to 0.0013% by weight of the total water weight of oleoresin capsicum;

0.030% by weight of the total water weight of chaste berry;

0.10% by weight of the total water weight of wild yam root;

0.025% by weight of the total water weight of fennel;

0.10% by weight of the total water weight of passion flower;

0.030% by weight of the total water weight of don quai extract;

0.025% by weight of the total water weight of spearmint oil;

0.05% by weight of the total water weight of grapefruit seed extract;

3.65% by weight of the total water weight of vegetable glycerin;

0.83% by weight of the total water weight of ascorbic acid; and

0.26% by weight of the total water weight of citric acid.

### EXAMPLE 7

Another example of the present invention relates to a prostate support composition for use with the intranasal drug delivery system. The prostate support composition provides support to the prostate and maintains the health of the prostate while improving blood flow to the prostate gland and penis. In addition, the prostate composition flushes out impurities from within the prostate gland while fortifying its chemical processes. The prostate support composition includes oleoresin capsicum including capsaicin, dihydrocapsaicin, nordihydrocapsaicin, homodihydrocapsaicin, and homocapsaicin as the intranasal drug delivery system; saw palmetto, pygeum africanum, and Chinese ginseng as the drug; and maitake mushroom extract, pine needle oil, spearmint oil, grapefruit seed extract, vegetable glycerin, ascorbic acid, and citric acid as other components.

The saw palmetto, pygeum africanum, and Chinese ginseng are used to maintain the health of the prostate by flushing out natural contaminants. The saw palmetto in particular, rejuvenates the production of chemicals that are lacking in patients with swollen prostate symptoms. Both the pygeum and ginseng increase blood flow and the natural chemical processes of a healthy prostate gland. The maitake mushroom extract provides immune system support, and high amounts of zinc necessary for replacement of low zinc levels in swollen prostate glands. The grapefruit seed extract is an anti-bacterial agent and preservative. The spearmint oil provides a sweet taste. The vegetable glycerin is a moisturizer, assists the capsaicinoids contained in the oleoresin capsicum to maintain its potency and effectiveness for longer periods of time, shortens the length of time of the burning sensation associated with the capsaicinoids in the oleoresin capsicum without reducing its effectiveness, and stabilizes the formula. The ascorbic acid adjusts the pH level, supports the immune system, and acts as a natural preservative. The citric acid adjusts the pH level and stabilizes the formula.

In another embodiment of the present invention, the prostate support composition may be homeopathic wherein the saw palmetto, pygeum africanum, and Chinese ginseng provide support to the prostate. Further, the homeopathic prostate support composition can include saw palmetto, pygeum africanum, and Chinese ginseng as tinctures.

In still another embodiment of the present invention, the prostate support composition may include between about 0.0023% to 0.0026% by weight of the total water weight of oleoresin capsicum including capsaicin, dihydrocapsaicin, nordihydrocapsaicin, homodihydrocapsaicin, and homocapsaicin for use with the intranasal drug delivery system.

Another embodiment of the prostate support composition for use with the intranasal drug delivery system in 5 gallons of purified water includes:

0.0023% to 0.0026% by weight of the total water weight of oleoresin capsicum;

0.20% by weight of the total water weight of saw palmetto;

0.026% by weight of the total water weight of pygeum africanum;

0.026% by weight of the total water weight of Chinese ginseng;

0.20% by weight of the total water weight of maitake mushroom extract;

0.010% by weight of the total water weight of pine needle oil;

0.010% by weight of the total water weight of spearmint oil;

0.05% by weight of the total water weight of grapefruit seed extract;

3.65% by weight of the total water weight of vegetable glycerin;

0.83% by weight of the total water weight of ascorbic acid; and

0.26% by weight of the total water weight of citric acid.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

1-24. (canceled)

25. An intranasal composition comprising
purified water; and
a water-soluble oleoresin capsicum including capsaicin, dihydrocapsaicin, nordihydrocapsaicin, homodihydrocapsaicin, and homocapsaicin,
wherein the oleoresin capsicum is about 0.000001% to 0.0071% by weight of the total weight of the purified water in the system.

26. The intranasal composition of claim 25, and further comprising
a drug,
wherein the water-soluble oleoresin capsicum acts as a carrier of the drug and increases the permeability of nasal membranes to the drug.

27. The delivery system in accordance with claim 26, wherein the drug is in liquid form.

28. The intranasal composition of claim 25, wherein the oleoresin capsicum is between about 1 ppb to 50,000 ppb of the system.

29. The intranasal composition of claim 25, wherein capsaicin, dihydrocapsaicin, nordihydrocapsaicin, homodihydrocapsaicins, and homocapsaicin have a heat level of about 100,000 to about 500,000 Scoville units.

30. The intranasal composition of claim 26, wherein the drug is homeopathic.

31. The intranasal composition of claim 25, wherein the water-soluble oleoresin capsicum comprises between about 67-71% capsaicin, about 20-24% dihydrocapsaicin, about 5-9% nordihydrocapsaicin, about 0.25-2% homodihydrocapsaicin, and about 0.25-2% homocapsaicin.

32. The intranasal composition of claim 26, wherein the drug comprises:
0.13% by weight of the total suspension material weight of eucalyptol;
0.10% by weight of the total suspension material weight of feverfew extract; and
0.0027% by weight of the total suspension material weight of peppermint oil, and
wherein the drug delivery system is effective to relieve at least one symptom associated with headaches or migraines.

33. The intranasal composition of claim 32, and further comprising:
0.0044% to 0.0047% by weight of the total water weight of water-soluble oleoresin capsicum;
0.08% by weight of the total water weight of rosemary extract;
0.05% by weight of the total water weight of grapefruit seed extract;
3.65% by weight of the total water weight of vegetable glycerin;
0.53% by weight of the total water weight of sea salt;
0.83% by weight of the total water weight of ascorbic acid; and
0.26% by weight of the total water weight of citric acid.

34. The intranasal composition of claim 26, wherein the drug comprises 0.13% by weight of the total water weight of eucalyptol; and 0.10% by weight of the total suspension material weight of nettle.

35. The intranasal composition of claim 34, and further comprising:
0.0029% to 0.0032% by weight of the total water weight of oleoresin capsicum;
0.09% by weight of the total water weight of rosemary extract;
0.05% by weight of the total water weight of grapefruit seed extract;
3.65% by weight of the total water weight of vegetable glycerin;
0.53% by weight of the total water weight of sea salt;
0.83% by weight of the total water weight of ascorbic acid; and
0.26 by weight of the total water weight of citric acid.

36. The intranasal composition of claim 26, and further comprising 0.20% by weight of the total water weight of licorice root extract; and 0.15% by weight of the total water weight of Chinese ginseng.

37. The intranasal composition of claim 36, and further comprising:
0.0018% to 0.0021% by weight of the total water weight of the total water weight of oleoresin capsicum;
0.20% by weight of the total water weight of green tea extract;
0.05% by weight of the total water weight of grapefruit seed extract;
3.65% by weight of the total water weight of vegetable glycerin;
0.025% by weight of the total water weight of spearmint oil;
0.83% by weight of the total water weight of ascorbic acid; and
0.26% by weight of the total water weight of citric acid.

38. The intranasal composition of claim 26, wherein the drug comprises 0.120% by weight of the total water weight of echinacea extract; 0.13% by weight of the total water weight of eucalyptol; and 0.15% by weight of the total water weight of golden seal extract based on the total water weight of the base.

39. The intranasal composition of claim 38, and further comprising:
0.0024% to 0.0027% by weight of the total water weight of oleoresin capsicum;
0.10% by weight of the total water weight of green tea extract;
0.10% by weight of the total water weight of cats claw extract;
0.013% by weight of the total water weight of spearmint oil;
0.05% by weight of the total water weight of grapefruit seed extract;
0.20% by weight of the total water weight of maitake mushroom extract;
3.65% by weight of the total water weight of vegetable glycerin;
0.53% by weight of the total water weight of sea salt;
0.83% by weight of the total water weight of ascorbic acid; and
0.26% by weight of the total water weight of citric acid.

40. The intranasal composition of claim 26, wherein the drug comprises 0.10% by weight of the total water weight of marshmallow root extract; and 0.15% by weight of the total water weight of kava extract.

41. The intranasal composition of claim 40, and further comprising:
0.0029% by weight of the total water weight of oleoresin capsicum;
0.0-26% by weight of the total water weight of spearmint oil;
0.05% by weight of the total water weight of grapefruit seed extract;
3.65% by weight of the total water weight of vegetable glycerin;
0.83% by weight of the total water weight of ascorbic acid; and
0.26% by weight of the total water weight of citric acid.

42. The intranasal composition of claim 26, wherein the drug comprises 0.030% by weight of the total water weight of chaste berry; 0.10% by weight of the total water weight of wild yam root; 0.025% by weight of the total water weight of fennel; and 0.10% by weight of the total water weight of passion flower.

43. The intranasal composition of claim 42, and further comprising:
0.0010% to 0.0013% by weight of the total water weight of oleoresin capsicum;
0.30% by weight of the total water weight of don quai extract;
0.025% by weight of the total water weight of spearmint oil;
0.05% by weight of the total water weight of grapefruit seed extract;
3.65% by weight of the total water weight of vegetable glycerin;
0.783% by weight of the total water weight of ascorbic acid; and
0.26% by weight of the total water weight of citric acid.

44. The intranasal composition of claim 26, wherein the drug comprises 0.20% by weight of the total water weight of saw palmetto; 0.026% by weight of the total water weight of pygeum africanum; and 0.026% by weight of the total water weight of Chinese ginseng.

45. The intranasal composition of claim 44, and further comprising:
0.0023% to 0.0026% by weight of the total water weight of oleoresin capsicum;
0.020% by weight of the total water weight of maitake mushroom extract;
0.010% by weight of the total water weight of pine needle oil;
0.010% by weight of the total water weight of spearmint oil;
0.05% by weight of the total water weight of grapefruit seed extract;
3.65% by weight of the total water weight of vegetable glycerin;
0.783% by weight of the total water weight of ascorbic acid; and
0.26% by weight of the total water weight of citric acid.

46. A method of delivering a drug to a human, comprising the steps of:
intranasally administering the composition according to claim 25 in combination with a drug,
wherein the water-soluble oleoresin capsicum acts to increase the permeability of nasal membranes to the drug.
